(19) United States
(12) Patent Application Publication
(10) Pub. No.: US 2008/0009441 A1
(43) Pub. Date: Jan. 10, 2008

(54) Polypeptide Inducing the Secretion of Angiopoietin

(75) Inventors: Yang-Je Cho, Seoul (KR); Bo-Young Ahn, Seoul (KR); Doo-Sik Kim, Seoul (KR); Won-Il Yoo, Gyeonggi-do (KR)

Correspondence Address: BAKER & DANIELS LLP, 205 W. JEFFERSON BOULEVARD, SUITE 250, SOUTH BEND, IN, 46601, US

(73) Assignee: EYEGENE, INC., SEOUL, KOREA, REPUBLIC OF 120-113 (KR)

(21) Appl. No.: 10/599,465

(22) PCT Filed: Mar. 31, 2004
(86) PCT No.: PCT/KR04/00751

§ 371 Date: Aug. 17, 2007

(51) Int. Cl. A61K 38/16; A61P 27/00; A61P 9/00; C07K 14/00

(52) U.S. Cl. 514/12; 530/324

(57) ABSTRACT

This invention relates to Cell supernatant protein for inducing angiogenesis-1 secretion. The protein of the invention can be useful as therapeutic agent for angiogenesis-related diseases.

### TECHNICAL FIELD

The present invention relates to a polypeptide inducing the secretion of angiopoietin which is effective on inhibition of abnormal angiogenesis. The polypeptide can be used as a therapeutic agent for treating diabetic retinopathy, immature infant retinopathy and so on.

### BACKGROUND ART

Generally, angiogenesis is referred to as a process that a sprout is generated from the existing microvessel and then grows into new capillaries. It is a very important and normal process for differentiation of embryo, amniotic fluid of uterine, growth of placenta, luteogenesis and wound healing (Gunther G. et al., Oncology 54:177-184 (1997), incorporated herein by reference to it). There are a variety of diseases associated with angiogenesis that grow abnormally, or neovascularization itself that may be caused by its abnormally controlled growth to become etiology. Examples of the disease include angiogenesis-related ocular diseases, rheumatic arthritis, any complications related to diabetes, psoriasis, pyogenic granuloma and so on.

Angiogenetic mechanism must be turned off in the normal physiological level of an eyeball. But when the mechanism is turned on by erroneous signaling, severe ocular diseases are suffered, causing a loss of eyesight (Lois E. H. et al., Nat Ned. 5:1390-1395 (1999)). Exemplary angiogenesis-related ocular diseases include diabetic retinopathy wherein blood vessel is formed in a retina, immature infant retinopathy, and age-related macular degeneration wherein blood vessel is formed in a choroid (Amal A. E. et al., Retina 11:244-249 (1991);Constantin J. P. et al., Ophthalmology 97:1329-1333 (1990); Jin-Hong C. et al., Current opinion in Ophthalmology 12:242-249(2001); and Peter A. C., J of Cellular Physiology 184:301-310(2000)). Immature infant retinopathy (ROP) known to cause most of infant blindness proceeds in two steps. Premature infants have an incomplete retinal blood vessel at the beginning of a birth, especially the premature infants who surfer from the progress of ROP have a risk of inducing no growth of blood vessel in a retina (Flynn J. T. et al., Arch Ophthalmol 95:217-223 (1977)). As a result, the retina is formed in a blood vessel-free state, resulting in formation of a low-oxygen peripheral retina (step 1 of ROP). In such step 1 of ROP, a non-perfusion level of retina determines a destructive stage including a retinal detachment and blindness caused by angiogenesis (step 2 of ROP) (Penn J. S. et al., Invest Ophthalmol Vis Sci 35:3429-435 (1994)). If blood vessel is normally developed in the retina of the premature infants, then a destructive stage may not be initiated due to a secondary angiogenesis in ROP. It has been known that use of high concentration of oxygen is associated with such diseases, which means that an oxygen-regulated factor is present in the retina of premature infants. It is anticipated that VEGF, which is necessarily required to a normal angiogenesis and known as a oxygen-regulated factor, should take a important role in ROP, but it is known from the various studies that VEGF act mainly in the first and secondary stage of ROP (Pierce E.A. et al., Arch Ophthalmol 114:1219-1228 (1996)). It was studied that VEGF expression is inhibited in the first stage to affect the growth of blood vessel, using ROP animal model (for example, high supplement oxygen). Diabetic retinopathy is one of the most well known conditions among microvessel-related complication mainly caused by hyperglycemia, and become a primary cause of acquired loss of sight in the adult (Brownlee M., Nature 414:813-820 (2001)). A serious loss of sight associated with diabetic retinopathy is generated by means to retinal angiogenesis (Battegay E.J., J Mol Med 73:333-346 (1995)) and therefore vitreous hemorrhage and 4 tractional retinal detachment (Cai J., Boulton M., Eye 16:242-260(2002)). Referring to a pathophysiological change in the retina of diabetic patients, the conditions such as loss of cells surrounding capillary vessel, growth of basement membrane, loss of automatic control function in retinal blood vessel, abnormality of capillary circulation, microaneurysm, IRMA (intraretinal microvascular abnormalities) have appeared, finally resulting in formation of an area of retinal non-perfusion (Lip P. L. et al., Invest Ophthalmol Vis Sci 41:2115-2119 (2000); Hammes H. P. et al., Diabetes 51:3107-3112 (2002)). Such changes induce an increased vascular permeability, chronic retinal hypoxia and retinal ischemia through their continuous development to form macular edema or angiogenesis, resulting in progress into proliferative diabetic retinopathy (Aiello L. P. et al., Diabetes Care 21:143-156 (1998)). It seems that diabetic patients have an increased level of a factor VEGF, and then the increased factor induces a retinopathy by destroying a retinal blood barrier. Age-related marcular degeneration is one of the major causes of blindness which appears over 50 years old. Severe loss of sight results from angiogenesis induced from capillary vessel of a choroidal neovascular membrane (Ferris F. L. 3rd et al., Arch Ophthalmol 102:1640-1642 (1984)). AMD is generally divided in 2 different types, for example wet AMD and dry AMD. It was known that development of wet AMD was followed by dry AMD. Dry AMD is referred to as the presence of macular degeneration due to pigmentary degeneration of retina and loss of retinal pigment epithelium (RPE). As the modified form of dry AMD, wet AMD shows conditions of subretinal neovascularization (subretinal scar), subretinal hemorrhage, detachment of RPE. In fact, subretinal neovascularization is meant to be a growing cicatricial tissue for a treatment of a space resulting from diseased RPE. Growth of neovascularization allows plasma and cellulose to be extruded therefrom, causing a small retinal detachment (Mousa S.A. et al., J Cell Biochem 74:135-43 (1999)). In addition, an injury caused by cicatrix of subretinal membrane may also result in weak eyesight.

Now, the method used to treat such ocular diseases includes laser treatment, laser photocoagulation, cryocoagulation and Visudyne (Edwin E. B. et al., Ophthalmology 88:101-107 (1981)). All of such treatments are carried out by surgery, but treatment by therapeutic agents still remains to be developed. Treatment by surgery has significant problems of incapable to be applied to all patients, and it also has disadvantages of having low healing possibilities and very expensive cost. Accordingly, most of patients, who may not receive a surgery, may come to blindness due to the lack of specific therapeutic agents. Also as human lives longer, these conditions continue to increase, but the therapeutic agents still remain to be developed. Thus, many studies and developments of-angiogenesis inhibitors and therapeutic agents for treating the ocular diseases are still carried out. And examples of such agents include steroids, MMP inhibitor, antibodies against angiogenic growth factor and so on (Jeremy G. et al., Am J Pathology 160:1097-1103(2002)).

Therefore, it is possible to treat such angiogenesis-related diseases by removing angiogenesis-inducing causes. That is to say, it is possible to treat the angiogenesis-related diseases by reinforcing the existing structure of blood vessel to fundamentally remove the angiogenesis-inducing causes. The reinforcement of the structure of blood vessel may prevent secondary ischemic condition and hence angiogenesis by destruction of blood vessel.

As the alternative method, attention is taken to a use of angiopoietin- 1 because it plays a role in stabilizing blood vessel (Nat Med 2000 Apr;6(4):460-3) and angiogenesis of VEGF. It has been reported that this mechanism was used to treat diseases such as retinopathy caused by peripheral vascular deficits by chronic diabetes, or immature infant retinopathy caused by deficits of normal formation of blood vessel (Am J Pathol.2002 May;160(5):1683-93). But, recombinant angiopoietin-1 may not be directly used in human due to problems of stability and solubility. As a alternative, materials showing the same activity as angiopoietin-1 remain to be developed (Exp Mol Med. 2002 Mar 31;34(1):1-l 1), and secretory materials of angiopoietin-1 also remain to be studies.

### DISCLOSURE OF INVENTION

Therefore, the present invention is designed to solve the problems of the prior art, and it is an object of the present invention to provide a therapeutic agent for inducing angiopoietin-1 secretion to facilitate a formation of a normal structure of blood vessel.

In order to accomplish the above object, the present invention provides a protein for inducing secretion of angiopoietin-1 expressed by amino acid sequence of SEQ ID NO 1. It also provides a therapeutic agent for inducing angiopoietin-1 secretion to stabilize angiogenesis and peripheral blood vessel.

As described here, the protein for inducing angiopoietin-1 secretion comprises a protein of SEQ ID NO 1, a fragment and variants having the same function of the protein of SEQ ID NO 1.

Angiogenesis-related diseases, which may be prevented and treated by the protein of the present invention, are preferably conditions which have a mechanism for inducing angiopoietin-1 secretion to facilitate a stabilization of angiogenesis, for example selected from the group consisting of pulmonary hypertension (Ann Thorac Surg 2004 feb 77(2) 449-56), ischemic myocardium (acting together with VEGF) (Biochem Biophys Res Common. 2003 Oct 24;310(3):1002-9), skin flap survival (Microsurgery. 2003;23(4):374-80), heart failure (Cold Spring Harb Symp Quant Biol 2002;67:417-27), acute hindlimb ischemia (acting together with VEGF) (Life Sci 2003 Jun 20;73(5):563-79) and so on. Ocular diseases are more preferred.

Ocular diseases capable to be used in the present invention are, in particular, selected from the group consisting of immature infant retinopathy, diabetic retinopathy and so on.

The present inventors have firstly found that the cancer metastasis inhibitor saxatilin induces angiopoietin-1 secretion. By using angiopoietin secretion in the two types of cell lines and ROP mouse model, we have also confirmed that abnormal angiogenesis-related disorders are treated with saxatilin.

They have firstly found that angiopoietin-1 secretion was induced in two cell lines if purely purified recombinant saxatilin was administered in a concentration-dependant manner. In the O₂ partial pressure animal model, it also was found that this mechanism aids to form a normal blood vessel without inhibiting a normally developing vascularization in the developmental stage, and reduces blood leakage from blood vessel of a morbid angiogenesis by stabilizing the structure of blood vessel, the morbid angiogenesis having a property of abnormal structure of blood vessel.

Accordingly, the present invention is preferably used in immature infant retinopathy which appears from normal developmental inhibition process of blood vessel, diabetic retinopathy associated with a abnormal neovascularization induced by destruction of a normal structure of blood vessel, and age-related macular degeneration etc.

### BRIEF DESCRIPTION OF THE DRAWINGS

These and other features, aspects, and advantages of preferred embodiments of the present invention will be more fully described in the following detailed description, taken accompanying drawings. In the drawings:

FIG. 1 is an electrophoretic photograph showing that a large amount of angiopoietin-1 is secreted from a fibrosarcoma cell line treated with saxatilin;

FIG. 2 is a photograph showing angiopoietin-l secretion from a 298T cell line treated with saxatilin;

FIG. 3 is an operating microscopic photograph showing that saxatilin peritoneally administered (10 ng - 1 ug/kg/day) facilitates retinal angiogenesis of mouse induced by VEGF;

FIG. 4 is a photograph showing that normal angiogenesis is facilitated, but abnormal angiogenesis is suppressed in the concentration-dependant manner by saxatilin peritoneally administered in the animal model for inducing retinal angiogenesis, by decreasing to normal O₂ partial pressure after high-pressure oxygen (75%) treatment; and

FIG. 5 is a photograph showing that blood leakage of blood vessel is reduced by saxatilin peritoneally administered in the animal model for inducing retinal angiogenesis by decreasing to normal O₂ partial pressure after high-pressure oxygen (75%) treatment, the photograph observed by using a phosphor FICT-dextran.

### BEST MODES FOR CARRYING OUT THE INVENTION

Hereinafter, preferred embodiments of the present invention will be described in detail with reference to the accompanying drawings.

### EXAMPLE 1

### Angiopoietin-1 Secretion in the Saxatilin-Treated Fibrosarcoma Cell Lines

Fibrosarcoma Cell Culture

Fibrosarcoma cell (human) was cultured at 37° C. in MEM supplemented with 10% FBS in the 5% CO₂ incubator. And the cell was used when at least 90% of the cell was grown in the petri dish.

Measurement of Angiopoietin-1 Secretion

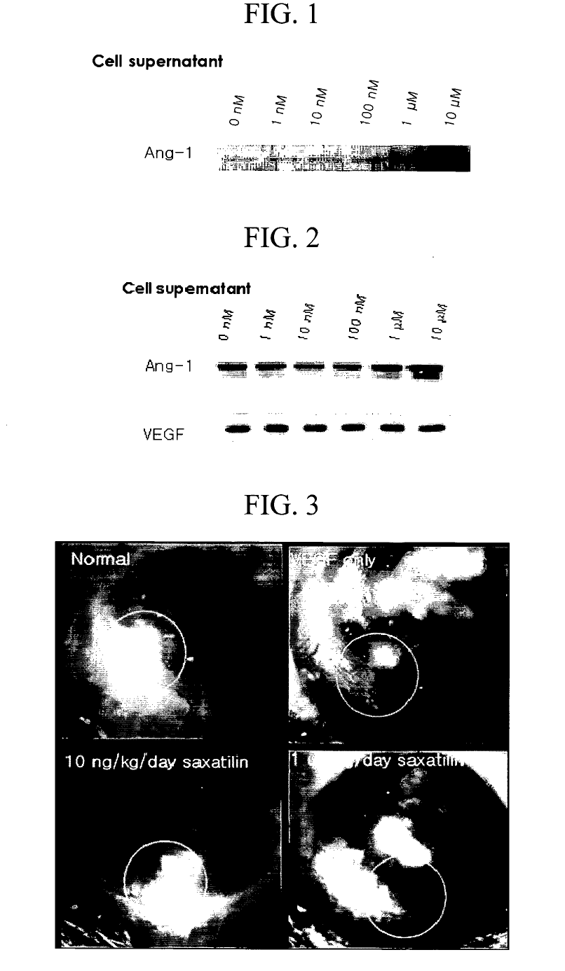

The cultured fibrosarcoma cell was treated with 0-10 ug of saxatilin to allow the cell to be a 2×10⁵ density in 6 well plates. After the saxatilin treatment, angiopoietin-1 secretion was induced for 12 hrs, and then the obtained amount of angiopoietin-1 was determined by western blotting (FIG. 1).

### EXAMPLE 2

### Angiopoietin-1 Secretion in the Saxatilin-Treated 298T Cell Lines

298T Cell Culture

298T cell (human) was cultured at 37° C. in MEM supplemented with 10% FBS in the 5% CO₂ incubator. And the cell was used when at least 90% of the cell was grown in the petri dish.

Measurement of Angiopoietin-1 Secretion

The cultured fibrosarcoma cell was treated with 0-10 ug of saxatilin to allow the cell to be a 2×10⁵ density in 6 well plates. After the saxatilin treatment, angiopoietin-1 secretion was induced for 12 hrs, and then the obtained amount of angiopoietin-1 was determined by western blotting (FIG. 2).

### EXAMPLE 3

### Effect of Saxatilin on VEGF-Induced Angiogenesis in a Blood Vessel-Free Corneal Tissue of the Eyeball

To investigate an effect of saxatilin on angiogenesis in the eyeball, an animal model was designed to create a micro pocket within cornea of the mouse eye, and insert a pellet containing 300 ng of VEGF to induce angiogenesis. At this time, 1 ug/kg of saxatilin was peritoneally administered so as to test an effect of saxatilin. 5 days after saxatilin administration, angiogenesis was observed in the eye of mouse using a stereo-microscope. As a result, it was found that peritoneal administration of saxatilin induced the proliferation of neovascularization without inhibiting growth of neovascularization (see FIG. 3).

In addition, side effects such as corneal opacity were not observed in the mouse eye used in the present experiment.

### EXAMPLE 4

### Effect of Saxatilin in the Mouse Model for Inducing Retinal Angiogenesis by change of O₂ Partial Pressure

It seems that artificial retinal angiogenesis caused by difference of 02 partial pressure has a similar aspect to immature infant retinopathy and diabetic retinopathy. The present experiment was carried out using a principle that if a mouse was exposed to 75% of a high-oxygen condition at the beginning of birth, and returned to 20% of a normal O₂ partial pressure, then abnormal angiogenesis was spontaneously induced in the mouse eye (Higgins R D. et al., Curr. Eye Res. 18:20-27 (1999); Bhart N. et al., Pediatric Res. 46:184-188 (1999); Gebarowska D. et al., Am. J. Pathol. 160:307-313 (2002)). For this purpose, 7 days after a mouse was borne in a device capable to control an O₂ partial pressure, the mouse was placed for 5 days under the high-oxygen condition having a constant 75% O₂ partial pressure, and then placed for 5 days under the 20% O₂ partial pressure. At this point, saxatilin was peritoneally administered once per day for 5 days, and then retinal angiogenesis was observed. To investigate whether blood vessel was formed in the eyeball, a solution was firstly prepared by dissolving 50 mg FITC-dextran (molecular weight: 2×10⁶) in 1 ml saline. The resulting solution was then administered through a left ventricle. The eyeball was extracted from the mouse immediately after the administration. The extracted eyeball was washed with saline, and fixed for 4-24 hrs with 4% paraformaldehyde. A lens was then removed from the eyeball, a retina was evenly placed on a glass slide, and the resulting glass slide was sealed with glycerin-gelatin, and then observed using fluorescent microscope.

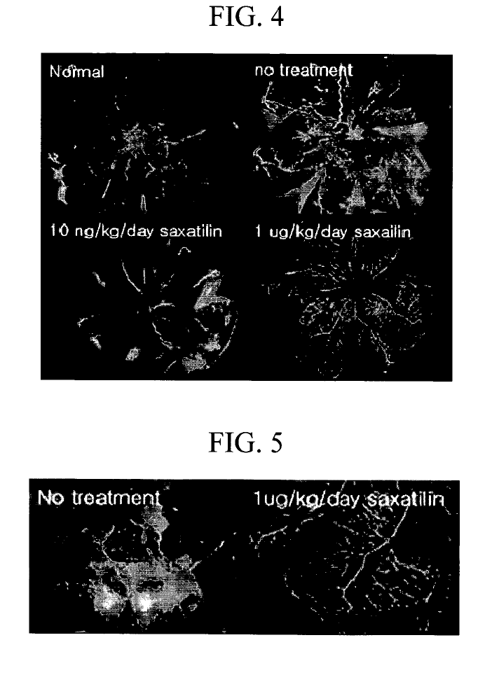

The conventional animal experiment of mouse was carried out on the basis of the amount of administered saxatilin (1 mg/kg/day) showing an efficacy of anti-cancer drug. As a result, it was found that plenty of neovascularization was formed around periphery of the retina in the mouse treated with the saline after exposure of high-pressure oxygen condition, while vascular tissues in development stage was not normally developed in the infant mouse placed only under the high-pressure oxygen condition, compared to a mouse which grow in the normal condition. However, it was seen that abnormal neovascularization was not observed in the mouse treated with 100 ng-100/μg/kg/day of saxatilin, and that a normally developed blood vessel was formed in the dose-dependant manner (see FIG. 4). Interestingly, it is seen that saxatilin may be used as a therapeutic agent regarding ocular diseases in that it has no effect on normal blood vessel, as well as playing a role in facilitating its growth. It seems that said result comes from angiopoietin-1 secretion by saxatilin. Accordingly, saxatilin may be used to treat immature infant retinopathy, because it showed an ability of inhibiting morbid angiogenesis by secreting angiopoietin-1 to reduce a low oxygen region and then removing causes of inducing angiogenesis, using the mouse model for inducing retinal angiogenesis by the change of O₂ partial pressure. In the mouse model, it was seen that inducing normal angiogenesis by angiopoietin-1 secretion was more effective than preventing abnormal angiogenesis in immature infant retinopathy. In addition, it was observed from the FITC-dextran fluorescence leakage test that blood leakage did not appeared because the structure of blood vessel was stabilized by treatment of a low dose of saxatilin (see FIG. 5). Large molecules are easily not leaked out from the retinal blood vessel due to the presence of blood-retina-barrier (BRB) such as blood-brain-barrier (BBB) of the cerebrovascules. It was also demonstrated from the present experiment that leakage of a relatively high molecular weight of FITC-dextran from the retina means that there are significant damages in the fine structure of the retinal blood vessel, and the injury was healed by angiopoietin-1 secretion by saxatilin.

Accordingly, saxatilin may be used as the therapeutic agent against these diseases such as diabetic retinopathy and age-related macular degeneration, because saxatilin aids to maintain the structure of blood vessel at the early stage (for example, angiogenesis does not occur at this stage) of the diseases even when the diseases occur due to disorders such as blood leakage from blood vessel.

### Industrial Applicability

The present invention is provided with a novel method for treating angiogenesis-related ocular diseases by using the therapeutic agents instead of the conventional surgeries. Treatment by surgery has problems of expensive cost and therefore inapplicability to all patients. However, the method according to the present invention is one of the methods for treating the angiogenesis-related ocular diseases to prevent the blindness. Secretion of angiopoietin-1 by saxatilin has not affect the existing normal blood vessel and the normal neovascularization to be newly formed in a developmental stage. On the contrary, angiopoietin-1 secretion gives significant advantages to the patients who suffer from the disorders of the developmental stage, such as immature infant retinopathy. It may be impossible to use saxatilin to treat immature infant retinopathy if the entire neovascularization is inhibited by saxatilin. Accordingly, saxatilin may be useful as a therapeutic agent for treating immature infant retinopathy. It is also fundamentally possible to treat immature infant retinopathy by preventing the structure of blood vessel at the beginning of it. And it seems that saxatilin inhibits an abnormal growth of blood vessel by aiding to normalize the structure of blood vessel in the age-related macular degeneration.

The present invention has been described in detail. However, it should be understood that the detailed description and specific examples, while indicating preferred embodiments of the invention, are given by way of illustration only, since various changes and modifications within the spirit and scope of the invention will become apparent to those skilled in the art from this detailed description.

What is claimed is:

1. A protein for inducing an angiopoietin-1 secretion, comprising an amino acid sequence depicted in SEQ ID NO 1.

2. A therapeutic agent for treating angiogenesis-related diseases, comprising an effective amount of the angiopoietin-1 secretion-inducing proteins comprising the protein of SEQ ID NO 1.

3. The therapeutic agent according to claim 2, wherein the angiogenesis-related diseases are selected from the group consisting of pulmonary hypertension, ischemic myocardium, skin flap survival, heart failure, acute hindlimb ischemia and ocular diseases.

4. The therapeutic agent according to claim 3, wherein the angiogenesis-related diseases are ocular diseases.

5. The therapeutic agent according to claim 3, wherein the angiogenesis-related diseases are selected from the group consisting of immature infant retinopathy, diabetic retinopathy and age-related macular degeneration.


SEQUENCE LISTING (1 sequence)

SEQ ID NO: 1 - protein, 73 aa, Agkistrodon saxatilis
EAGEECDCGAPANPCCDAATCKLRPGAQCAEGLCCDQCRFMKEGTICRMARGDDMDDYCNGISAGCPRNPFHA